(19) United States
(12) Patent Application Publication
(10) Pub. No.: US 2007/0053983 A1
(43) Pub. Date: Mar. 8, 2007

(54) Extended release compositions of metoprolol succinate

(76) Inventors: Girish Jain, Pitampura (IN); Mohan Kumar Kondapaturu, Hyderabad (IN); Utathya Bhadra, Kolkata (IN)

Correspondence Address: BIO INTELLECTUAL PROPERTY SERVICES (BIO IPS) LLC, 8509 KERNON CT., LORTON, VA, 22079, US

(21) Appl. No.: 11/339,676

(22) Filed: Jan. 26, 2006

(30) Foreign Application Priority Data
Sep. 6, 2005 (IN) 1084/MUM/2005

(51) Int. Cl. A61K 31/205; A61K 9/14

(52) U.S. Cl. 424/486; 424/488; 514/554

(57) ABSTRACT

The present invention relates to sustained release solid pharmaceutical composition comprising antihypertensives, in particular, Metoprolol succinate or pharmaceutically acceptable derivatives thereof and a process for preparing such a formulation. The present invention is a composition comprising Metoprolol succinate or its pharmaceutically acceptable derivatives thereof and the composition releases the drug over 24 hours. The composition further comprises hydrophilic polymer matrix based tablets. The present invention describes a sustained release tablet comprising sustained release matrix comprising of gelling agents comprising at least one hydrophilic polymer with one or more gum and gum derivatives.

### STATEMENT REGARDING FEDERALLY SPONSORED RESEARCH OR DEVELOPMENT

This invention has been created without the sponsorship or funding of any federally sponsored research or development program.

### CROSS REFERENCE TO RELATED APPLICATION

This application claims the benefit of Indian Patent Application No. 1084/MUM/2005, filed Sept. 06, 2005. The entire disclosure of this prior application is hereby incorporated by reference.

### SEQUENCE LISTING OR PROGRAM

Not applicable.

### BACKGROUND OF THE INVENTION

1. Field of Invention

The present invention relates to a sustained or modified release solid pharmaceutical composition comprising antihypertensives, in particular, Metoprolol succinate or pharmaceutically acceptable derivatives thereof and a process for preparing such a formulation.

2. Background of the Invention

β-blockers or β-adrenergic blocking agents are a class of drugs used to treat a variety of cardiovascular conditions and certain other diseases. β-blockers block the action of epinephrine and norepinephrine on the β-adrenergic receptors in the body (primarily in the heart, peripheral blood vessels, bronchi, pancreas, and liver). The hormones and neurotransmitters stimulate the sympathetic nervous system by acting on these receptors.

There are three types of beta receptors: β₁-receptors located mainly in the heart, and β₂-receptors located all over the body, but mainly in the lungs, muscles and arterioles. β₃-receptors are less well characterised, but have a role in fat metabolism.

Activation of β₁-receptors by epinephrine increases the heart rate and the blood pressure, and the heart consumes more oxygen. Drugs that block these receptors therefore have the reverse effect: they lower the heart rate and blood pressure and hence are used in conditions when the heart itself is deprived of oxygen. They are routinely prescribed in patients with ischemic heart disease. In addition, β-blockers prevent the release of renin, which is a hormone produced by the kidneys which leads to constriction of blood vessels.

Drugs that block β₂ receptors generally have a calming effect and are prescribed for anxiety, migraine, esophageal varices and alcohol withdrawal syndrome, among others. Many β-blockers affect both type 1 and type 2 receptors; these are termed non-selective blockers. Selective β-blockers primarily affect β₁-receptors.

The β-adrenergic blockers have an important role in the pharmacotherapy of ischemic heart disease, heart failure, arrhythmia, and hypertension. The β-adrenergic blockers vary in their lipid solubility, selectivity for the β₁-adrenergic receptor subtype, presence of partial agonist or intrinsic sympathomimetic activity, and membrane-stabilizing properties. Regardless of these differences, almost all of the β-adrenergic receptor antagonists are also equally effective as antihypertensive agents.

The β-blockers (examples: atenolol, metoprolol, propranolol) act as competitive antagonists at the adrenergic β₁ receptors. The newer agents tend to be more selective for the cardiac (β-1) receptors which allows for decreased systemic side effects.

β-adrenergic blockers effectively reduce the blood pressure of many patients with combine systolic and diastolic hypertension and of elderly patients with isolated systolic hypertension. The mechanism of the antihypertensive effects of β-blocking agents has not been elucidated. However, several possible mechanisms have been proposed: (1) competitive antagonism of catecholamines at peripheral (especially cardiac) adrenergic neuron sites, leading to decreased cardiac output; (2) a central effect leading to reduced sympathetic outflow to the periphery; and (3) suppression of rennin activity. Commonly used β-blockers include Acebutol, Atenolol, Betaxolol, Bisoprolol, Cartelol, Carvedilol, Esmolol, Labetolol, Metoprolol, Nadolol, Penbutolol, Pindolol, Propranolol, Timolol and the like.

Metoprolol is first selective β-adrenergic blocker devoid of intrinsic sympathomimetic activity and at higher plasma concentrations; metoprolol also inhibits β2-adrenoreceptors, chiefly located in the bronchial and vascular musculature. Metoprolol is a potent inhibitor of β-receptor mediated effects mainly involving β₁-adrenoreceptors. Such effects include not only reduction of exercise-induced tachycardia but also antihypertensive and cardiac antianginal and antiarrhythmic effects.

Clinical pharmacology studies have confirmed the β-blocking activity of metoprolol in man, as shown by (1) reduction in heart rate and cardiac output at rest and upon exercise, (2) reduction of systolic blood pressure upon exercise, (3) inhibition of isoproterenol-induced tachycardia, and (4) reduction of reflex orthostatic tachycardia.

Metoprolol is a secondary amine and is widely employed in the form of its succinate salt, namely (±) 1-(isopropylamino)-3-[p-(2-methoxyethyl)phenoxy]-2-propanol succinate.

Metoprolol succinate has the following structural formula

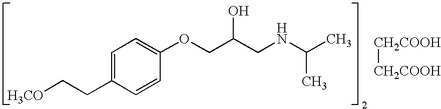

Metoprolol is a basic drug with pKa of 9.6. However it's succinate salt shows a pH in the range of 6-7 (2% w/v aqueous solution). The succinate salt is freely soluble in water.

The in vivo absorption of metoprolol is rapid and complete. The plasma metoprolol levels following administration of extended release metoprolol succinate are characterized by lower peaks, longer time to peak and significantly lower peak to trough variation. The peak plasma levels following once daily administration of modified release metoprolol succinate average one-fourth to one-half the peak plasma levels obtained following a corresponding dose of conventional metoprolol, administered once daily or in divided doses. At steady state the average bioavailability of metoprolol following administration of extended release metoprolol succinate, across the dosage range of 50 to 400 mg once daily, was 77% relative to the corresponding single or divided doses of conventional metoprolol.

Several formulations of β-adrenergic blockers have been reported in the literature, many of which relate to formulations of metoprolol. For example, formulations relating to oral, controlled release and pulse release compositions for metoprolol or its salts and derivatives have been reported. Examples of patents describing such formulations are as follows.

U.S. Pat. No. 4,957,745 assigned to Aktiebolaget Hassle describes a controlled release metoprolol. The preparation includes a plurality of beads comprising metoprolol coated with a polymeric membrane comprising ethyl cellulose with or without hydroxypropyl methylcellulose. Metoprolol or its salts such as tartrate, succinate or fumarate are used in the invention. The drug may be sprayed on the beads and then coated with polymers and finally filled into capsules or compressed as tablets. The process involves many steps and hence is complex and may not be preferred on commercial scale.

U.S. Pat. No. 4,871,549 assigned to Fujisawa Pharmaceuticals Inc., describes a time controlled explosion system comprising metoprolol, a swelling agent such as a low substituted hydroxypropyl cellulose, sodium starch glycolate or carboxymethyl cellulose sodium, coated with a water-insoluble coating material so that drug release is caused by the explosion of the membrane after a definite time period. This explosion of the outer membrane is caused by the power of swelling occurred when swelling agent absorbs the fluid. The specification illustrates metoprolol tartrate and other drugs such as metoclopramide. However, it does not discuss metoprolol succinate compositions.

U.S. Pat. No. 4,792,452 assigned to E. R. Squibb Inc. describes controlled release pharmaceutical compositions, which are said to provide pH-independent release for a basic drug such as Verapamil. The formulations include a pH-dependent polymer, which is a salt of alginic acid, a pH-independent hydrocolloid gelling agent and a binder. The salt of the alginic acid is preferably sodium alginate or potassium alginate. The weight ratio of the alginic acid salt to the hydrocolloid gelling agent is all within the range 0.1:1 to 10:1, and the formulation is free of calcium ion and carbon dioxide-producing material.

U.S. Pat. No. 5,081,154 assigned to Aktiebolaget Hassle is directed to metoprolol succinate in an oral composition coated with an anionic polymer soluble at pH over 5.5 and a water insoluble quaternary ammonium substituted acrylic polymer.

Further, U.S. Pat. Nos. 5,399,358 and 5,399,362, both the patents assigned to Edward Mendell Co. Inc., disclose a sustained release oral solid dosage form of metoprolol which includes a sustained release excipient including a gelling agent, an inert pharmaceutical diluent, and a cationic cross-linking agent. The sustained release component comprises of one heteropolysaccharide gum alongwith one homopolysaccharide gum capable of crosslinking with the heteropolysaccharide gum. The formulation provides release of metoprolol for at least about 24 hours.

US application 20030228361 (Penwest Pharmaceuticals Co) describes sustained release oral dosage forms of metoprolol tartrate. The said formulation comprises of metoprolol tartrate along with a sustained release excipient comprising a gelling agent comprising a heteropolysaccharide gum and a homopolysaccharide gum capable of cross-linking. The said heteropolysaccharide gum when exposed to environmental fluid and the said dosage form providing a mean Cmax of about 10-40 ng/ml per 100 mg metoprolol tartrate over 24 hours after oral administration. The said formulation is overcoated with a hydrophobic coating polymer. The invention describes xanthan gum as the heteropolysaccharide gum and locust bean gum as homopolysaccharide gum. The combination of these two as described in the invention exhibits synergism producing a higher viscosity and faster hydration than that which would be expected by either of the gums alone, the resultant gel being faster-forming and more rigid.

Yet, the rheological behavior of xanthan gum and locust bean gum systems form weak gels. These systems give maximum synergistic effect when the ratio of xanthan gum to locust bean gum is 1:1. Also, as the temperature increases, locust bean gum is reported to show downward shift of viscosity values. This direction of change in apparent viscosity may not be readily predictable with presence of other additives or polymers mixed with a gel and hence may affect the long-term stability of the formulation.

Metoprolol succinate is freely soluble in water and hence judicious selection of release retarding excipients is necessary to achieve a constant in vivo input rate of the drug. The most commonly used method of modulating the drug release is to include it in a matrix system.

Hydrophilic matrix systems are widely used in oral controlled drug delivery because of their flexibility to obtain a desirable drug release profile, cost-effectiveness, and broad regulatory acceptance. The drug release for extended duration, particularly for highly water-soluble drugs, using a hydrophilic matrix system is restricted due to rapid diffusion of the dissolved drug through the hydrophilic gel network. Upon administration, a rapid dissolution of the drug from the surface of the tablet is usually observed. The release rate of the drug from such systems is markedly influenced by the percentage and the type of the gum used. Liberation rate also depends on physical and chemical properties of the drug.

Therefore, despite the availability of different technologies for modified release preparation containing highly soluble drug such as metoprolol succinate, there is a clinical need for better modified released preparations with simple, stable, and easily manufactured compositions giving improved patient compliance, better and more uniform clinical effects and possible enhanced bioavailability.

To this end, present invention reports modified release composition of metoprolol succinate or its pharmaceutically acceptable derivatives thereof using hydrophilic polymer matrix comprising gelling agents comprising at least one hydrophilic polymer with one or more gum or gum derivatives and other suitable excipients to form the core which may optionally be coated with suitable polymers.

### SUMMARY OF THE INVENTION

The present invention provides novel sustained release compositions suitable for oral administration comprising at least one antihypertensive such as metoprolol succinate or its pharmaceutically acceptable derivative. The said composition further comprises metoprolol succinate or its pharmaceutically acceptable derivative along with a hydrophilic polymer matrix comprised of gelling agents comprising at least one hydrophilic polymer with one or more gum or gum derivatives thereof and other suitable pharmaceutically acceptable excipients. The said composition may optionally be coated with suitable pharmaceutical polymers. The said composition releases the drug over 24 hours.

Accordingly, it is an object of the present invention to provide an oral sustained or modified release pharmaceutical composition and a process for preparing the same for administration of antihypertensives to patients suffering from hypertension and other related disorders.

Another object of the present invention is to provide a sustained or modified solid pharmaceutical composition adapted for oral administration.

It is further object of the present invention to provide an oral sustained or modified release formulation of metoprolol succinate or pharmaceutically acceptable derivatives thereof suitable for once-a-day administration.

It is a further object of the present invention to provide oral sustained release composition, which release metoprolol succinate over a time period of at least about 24 hours when exposed to gastrointestinal tract.

Yet another object of the present invention is a sustained or modified metoprolol succinate composition comprised of metoprolol succinate or its pharmaceutical derivatives thereof in a core formed by the drug and hydrophilic polymer matrix comprised of gelling agents comprising at least one hydrophilic polymer with one or more gum or gum derivatives thereof.

The present invention, therefore also provides a pharmaceutical sustained or modified release formulation comprising at least one antihypertensive such as metoprolol succinate or pharmaceutically acceptable derivatives thereof along with hydrophilic polymer matrix comprised of gelling agents comprising at least one hydrophilic polymer with one or more gum or gum derivatives thereof and other suitable pharmaceutically acceptable excipients. This ‘core’ may optionally be coated with a suitable coating material.

The present invention further provides a method of administering to a subject in need of treatment a pharmaceutical product or formulation substantially as hereinbefore described and in particular a sustained or modified release composition which can be administered orally and as such is particularly suited for the treatment of hypertension and other related disorders.

The present invention therefore provides core comprised of metoprolol succinate or its pharmaceutically acceptable derivative thereof with hydrophilic polymer matrix comprised of gelling agents comprising at least one hydrophilic polymer with one or more gum or gum derivatives thereof and other suitable pharmaceutically acceptable excipients wherein the drug and the polymer and other suitable excipients are mixed together, granulated using suitable methods of granulation known in the art and then compressed together to yield tablets.

The present invention is further directed to a sustained release oral solid dosage form for absorption of metoprolol succinate or its pharmaceutically acceptable derivative thereof in the gastrointestinal tract, said drug comprising an effective amount of metoprolol succinate; and a hydrophilic polymer matrix comprised of gelling agents comprising at least one hydrophilic polymer with one or more gum or gum derivatives thereof when exposed to gastrointestinal fluid, said dosage form providing a therapeutic effect for about 24 hours after oral administration.

The present invention further provides use of at least one antihypertensive such as metoprolol succinate or its pharmaceutically acceptable derivative thereof in the manufacture of a medicament for the treatment of hypertension and other related disorders. Such a medicament according to the present invention comprises a sustained or modified release formulation substantially as hereinafter described.

Yet, another aspect of the present invention is the process of manufacturing the sustained or modified release composition. In particular, the invention provides a sustained or modified release composition comprising at least one antihypertensive such as metoprolol succinate or its pharmaceutically acceptable derivative thereof together with hydrophilic polymer matrix comprised of gelling agents comprising at least one hydrophilic polymer with one or more gum or gum derivatives thereof and other suitable pharmaceutically acceptable excipients. This ‘core’ may optionally be coated with a suitable coating material.

The present invention provides obvious benefits being simple and fast operational process for manufacturing said oral solid sustained release pharmaceutical composition.

Further aspects and embodiments of the invention may become apparent to those skilled in the art from a review of the following detailed description, taken in conjunction with the examples and the claims. It must be understood that that the present disclosure is intended as illustrative, and is not intended to limit the invention to the specific embodiments described herein.

### BRIEF DESCRIPTION OF THE DRAWINGS

FIG. 1 illustrates comparative dissolution profile of Metoprolol succinate from Metoprolol succinate sustained release tablets (Example-1-8) in pH 6.8 Phosphate Buffer

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a”, “an” and “the” include plural reference unless the context clearly dictates otherwise. Unless defined otherwise, all technical and scientific terms used herein have the same meaning as commonly understood to one of ordinary skill in the art to which this invention belongs.

Unless defined otherwise, all technical and scientific terms used herein have the same meaning as commonly understood to one of ordinary skill in the art to which the invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The information below is not admitted to be prior art to the present invention, but is provided solely to assist the understanding of the reader.

The details of one or more embodiments of the invention are set forth in the description and the examples below. Other features, objects, and advantages of the invention will be apparent from the description and from the claims.

The present invention provides novel sustained release compositions suitable for oral administration comprising at least one antihypertensive such as metoprolol succinate or its pharmaceutically acceptable derivative thereof. The said composition further comprises metoprolol succinate or pharmaceutically acceptable derivatives thereof along with hydrophilic polymer matrix comprised of gelling agents comprising at least one hydrophilic polymer with one or more gum or gum derivatives thereof and other suitable pharmaceutically acceptable excipients. The said composition may optionally be coated with suitable pharmaceutical polymers. The said composition releases the drug over 24 hours.

The term “pharmaceutically acceptable derivative” means various pharmaceutical equivalent isomers, enantiomers, complexes, hydrates, polymorphs, and etc. of metoprolol succinate.

The term composition includes but not limited to solutions and/or suspensions, dispersions, concentrates, ready mix, powders, granules, tablets, micro-tablets, capsules, pellets, comprising metoprolol succinate or pharmaceutically acceptable derivatives thereof in a core constituted by drug and hydrophilic polymer matrix comprising at least one hydrocolloid gelling agent which may be coated optionally using suitable coating material.

The term “therapeutically effective amount” means an amount of the drug, which is capable of eliciting a physiological response in a human patient. More specifically, the term “therapeutically effective amount” means the amount of drug, which is capable of treating hypertension and related disorders.

The term “sustained release” means that the therapeutically active medicament is released from the composition at a controlled rate such that therapeutically effective blood levels of the medicament are maintained over an extended period of time, e.g. providing a 24 hours therapeutic effect.

The term “gelling agents” means colloids in a more solid form than a sol. Usually gelling agent or gel forming agents are defined as semisolid systems consisting of either suspensions made up of small inorganic particles, or large organic molecules interpenetrated by a liquid. Gels consist of organic macromolecules uniformly distributed throughout a liquid in such a manner that no apparent boundaries exist between the dispersed macromolecules and the liquid. Gel-forming polymers produce materials that span a range of rigidities, beginning with a sol and increasing in rigidity to gel and hydrogel.

The medicament according to the present invention comprises a formulation substantially as herein described, and in particular a capsule, a tablet, micro-tablets, granules or pellets filled in capsule formulaton, typically a sustained or modified release tablet formulation substantially as hereinafter further described.

Suitably a formulation according to the present invention provides a novel sustained release dosage form, preferably tablets comprising core comprising metoprolol succinate or pharmaceutically acceptable derivatives thereof along with hydrophilic polymer matrix comprised of gelling agents comprising at least one hydrophilic polymer with one or more gum or gum derivatives thereof and other suitable pharmaceutically acceptable excipients and the said core optionally coated with suitable coating material and the process for preparing the same.

In a preferred embodiment of the present invention, a sustained release or modified release formulation comprises a pharmaceutically active agent (metoprolol succinate) along with suitable excipients. In particular, the present invention provides sustained release tablet formulations comprising a sustained release source of at least one antihypertensive such as metoprolol succinate. As such in a formulation according to the present invention, metoprolol succinate after oral administration can be released over a period of 24 hours. It has been observed that tablets according to the present invention produce relatively uniform blood levels of metoprolol succinate over extended periods of therapy, suitably with oral administration. A sustained/modified release is thus achieved by formulation substantially as hereinbefore described.

In a preferred embodiment of the present invention, a sustained release formulation comprises of at lease one pharmaceutically active agent, which may be formulated so that the release of the drug being held significantly pH-independent throughout the environment of the gastrointestinal tract.

The sustained release tablet dosage forms of metoprolol succinate according to the present invention may be formulated by mixing the drug with gelling agents comprising at least one hydrophilic polymer with one or more gum or gum derivatives thereof to form hydrophilic polymer matrix. In particular, in accordance with the present invention, a controlled release pharmaceutical formulation is provided from which an antihypertensive is released, at a controlled rate relatively independent of the pH of the environment such that in vivo consistent release is achieved throughout the gastrointestinal tract. The sustained release pharmaceutical formulation of the invention will preferably be in the form of a tablet and includes an antihypertensive such as metoprolol succinate or its pharmaceutically acceptable derivative thereof; a pH-dependent polymer which preferably is a water soluble salt of alginic acid with a pH-independent hydrophilic polymer together forming a hydrophilic polymer matrix along with suitable diluents and other excipients.

Polymers belonging to hydrophilic matrix systems, when exposed to an aqueous medium, does not disintegrate, but immediately after hydration develops a highly viscous gelatinous surface barrier which controls the drug release from and the liquid penetration into the center of the matrix system.

Therefore, it is theorized that upon oral ingestion of the sustained release tablet of the invention, in an acid aqueous environment, such as the stomach the pH-independent hydrophilic polymer hydrates to form a gel layer at the surface of the tablet. At this low pH environment sodium calcium alginate is formed in situ-formed by calcium carbonate and sodium alginate when added together, sodium alginate forms sodium calcium alginate in situ which acts as a pH independent system to release the drug in controlled manner; and this modifies the gel layer around the tablet, i.e. hydration and gelation of the alginate and cross-linking by calcium occurs to provide a gel barrier at the surface. Initially, there is a phase of rapid drug release but as gelation occurs, the release is controlled. Erosion of the gel layer gradually exposes more dry matrix that hydrates to replenish the gel layer. Drug dissolves in the gel layer and diffuses out into the surrounding aqueous environment.

As pH is increased, with passage of the tablet from the stomach down the gastrointestinal tract, the alginic acid salt in the tablet becomes more soluble and the alginic acid formed in the stomach will be reconverted to a more soluble salt, and it will structure the hydrophilic polymer gel layer less. Drug can diffuse more readily through the gel layer now and the ensuing increase in release rate from the matrix compensates for the reducing driving force for dissolution at the elevated pH values in GI tract further.

In another embodiment of the present invention, the hydrophilic polymer matrix is incorporated in the matrix along with the drug which matrix provides for the sustained release of metoprolol succinate or its pharmaceutically acceptable derivatives thereof.

In particular, the present invention provides sustained release tablets comprising a sustained release source comprising hydrophilic polymer matrix comprised of gelling agents comprising at least one hydrophilic polymer with one or more gum or gum derivatives thereof and other suitable pharmaceutically acceptable excipients such as diluents, binders, disintegrants, glidants, lubricants and the like.

Preferable examples of such hydrophilic polymers without any limitation include, cellulose polymers, in particular cellulose ethers such as methyl cellulose, cellulose alkyl hydroxylates such as hydroxypropylmethyl cellulose, hydroxypropyl cellulose, hydroxymethyl cellulose, hydroxyethyl cellulose, cellulose alkyl carboxylates such as carboxymethyl cellulose, and alkali metal salts of cellulose alkyl carboxylates, providone, vinyl copolymers, polyethylene oxide and derivatives thereof, propylene glycol esters, carbomers and derivatives thereof and the like, gums and gum derivatives such as vegetable gums such as alginates, xanthan gum, gum karaya, pectin, agar, tragacanth, acacia, carrageenan, tragacanth, chitosan, agar, alginic acid, other polysaccharide gums and mixtures thereof and carboxymethylether and propylene glycol esters, carbomers and derivatives thereof, polyvinyl pyrrolidones and derivatives thereof and the like.

In general, the present invention provides a process for the manufacture of a pharmaceutical product. The said composition is prepared by using wet granulation or dry granulation technique known in the art.

The process for preparing said composition comprises blending of metoprolol succinate or its pharmaceutically acceptable derivative thereof with suitable diluents, gelling agents comprising at least one hydrophilic polymer with one or more gum or gum derivatives and granulated using suitable granulator with or without the use of suitable binders and using aqueous or nonaqueous or hydroalcoholic solvents. The resultant granules are sized and blended with suitable disintegrants, release modifiers, glidants, lubricants and the like. The lubricated granules are then finally compressed on suitable compression machine. The tablets thus manufactured may optionally be coated to get quality product.

Alternatively, second process for preparing the said modified release composition comprises blending of metoprolol succinate or its pharmaceutically acceptable derivative thereof with suitable diluent, hydrophilic polymer, disintegrants, release modifiers, other polymers or binders, gum or gum derivatives, glidants and lubricants and the like and then compacting/compressing the same using suitable compactor/granulator. The compacts/granules are milled, sized and lubricated and then finally compressed on suitable compression machine. The tablets thus manufactured may optionally be coated to get quality product. More specifically, the present invention suitably the antihypertensive containing formulation core of a pharmaceutical composition according to the present invention may be formulated so as to allow the release of the drug there from in a still sustained or modified release manner, subsequent to the desired release profile provided by the inclusion of hydrophilic polymer matrix.

Suitable excipients employed in a pharmaceutical composition according to the present invention, may include commonly used pharmaceutical excipients such as pharmaceutically acceptable saccharide, including monosaccharides, disaccharides, polyhydric alcohols and/or mixtures thereof. Examples of suitable diluents include lactose, various forms of lactose, mannitol, sucrose, dextrose, microcrystalline cellulose, powdered cellulose, starches, sorbitol, dibasic calcium phosphate, calcium carbonate, magnesium oxide, and other mineral bases

Other commonly used pharmaceutical excipients would comprise of (a) binders, e.g. acacia, alginic acid, carbomer, carboxymethyl cellulose sodium, ethyl cellulose, guar gum, hydroxypropyl cellulose, hydroxypropylmethyl cellulose, hydroxyethyl cellulose, magnesium aluminum silicate, methyl cellulose, povidone, pregelatinized starch, sodium alginate, starch, dextrin, gelatin, hydrogenated vegetable oils, polymethacrylates, zein and the like; (b) lubricants (c) glidants; and (d) coatings and protective matrices, e.g. polymeric substances or waxes.

The formulation according to the present invention may also include pharmaceutically acceptable lubricants and glidants. The artisan can select appropriate lubricants and glidants in order to get good flow to aid in compression of the tablets.

Examples of suitable lubricants and glidants include magnesium stearate, calcium stearate, glyceryl behenate, light mineral oil, polyethylene glycol, sodium steryl fumarate, stearic acid, talc, hydrogenated castor oil, calcium silicate, magnesium silicate and colloidal silicon dioxide. The most preferred pharmaceutical lubricant and glidants are talc and magnesium stearate.

The present invention further comprises a process of preparing a pharmaceutical product, or a pharmaceutical composition, or a medicament substantially as hereinbefore described.

Substantially as hereinbefore described the tablets of the present invention comprise at least one antihypertensive such as metoprolol succinate or its pharmaceutically acceptable derivatives thereof, is prepared by blending metoprolol succinate with suitable diluents, at least one hydrophilic polymer and one or more gum or gum derivatives. The resultant product may then be granulated and the granules may then be sized and mixed and blended with disintegrants, glidants, release retardants, lubricants and the like. The resultant product may then be compressed and may optionally be coated using suitable polymers.

The present invention will now be illustrated with reference to the following examples, which does not limit the scope of the invention in any way. Further different strengths of the formulation may be achieved by proportionately using a dose weight scale-up or scale-down formula. The concentration of the excipients may also be varied or modified to achieve the desired dissolution profile by a skilled artisan.

### EXAMPLES

### Example—1

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 31 |
| 2. | Methocel K15 M CR | 150 | 24 |
| 3. | Hydroxypropyl cellulose (HPC-LF) | 50 | 9 |
| 4. | Microcrystalline cellulose (Avicel | 15 | 2.7 |
| | PH102) | | |
| 5. | Sodium Alginate (Keltone HVCR) | 80 | 14 |
| 6. | Calcium carbonate (Heavy) | 56 | 10 |
| 7. | Magnesium stearate | 5 | 0.9 |
| 8. | Talc | 9 | 1.6 |
| 9. | Purified water | q.s | q.s |

Procedure: Blend metoprolol succinate, methocel k 15 m, hydroxypropyl cellulose-L, microcrystalline cellulose, sodium alginate, calcium carbonate. The above blend was granulated with water. The resulting granulation was dried, milled and talc and magnesium stearate. The blended material was compressed using suitable compressing machine.

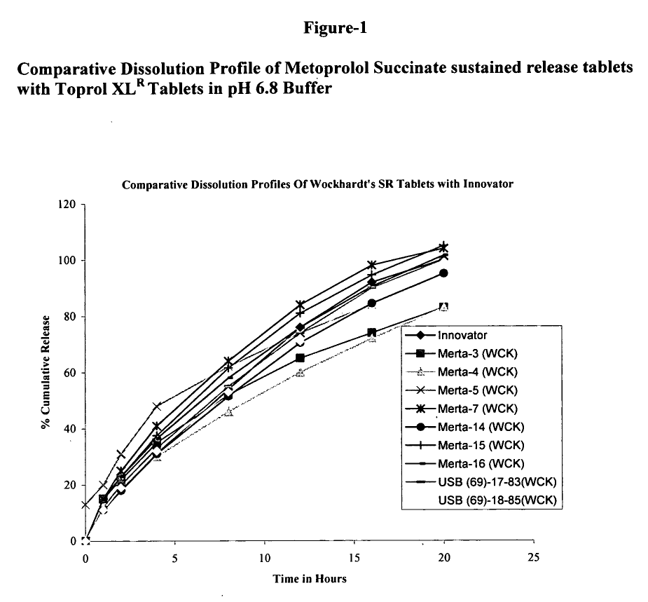

FIG. 1 shows the comparative dissolution profiles of the said invented tablet formulation and the reference formulation (Toprol XL Tablets).

### Example—2

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 29.7 |
| 2. | Lactose monohydrate | 50 | 7.8 |
| 3. | Methocel K15 M CR | 150 | 23.44 |
| 4. | Hydroxypropyl cellulose (HPC-LF) | 100 | 15.63 |
| 5. | Sodium Alginate (Keltone HVCR) | 80 | 12.5 |
| 6. | Calcium carbonate (Heavy) | 56 | 8.75 |
| 7. | Magnesium stearate | 5 | 0.78 |
| 8. | Talc | 9 | 1.41 |
| 9. | Purified water | q.s | q.s |

Procedure: Blend metoprolol succinate with lactose monohydrate. The above blend was granulated with water. The resulting granulation was dried, milled and blended with methocel, HPC, calcium carbonate, sodium alginate, talc and magnesium stearate. The blended material was compressed using suitable compressing machine.

FIG. 1 shows the comparative dissolution profiles of the said invented tablet formulation and the reference formulation (Toprol XL Tablets).

### Example—3

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 31.67 |
| 2. | Lactose monohydrate | 60 | 10 |
| 3. | Methocel K15 M CR | 150 | 25 |
| 4. | Hydroxypropyl cellulose (HPC-LH- | 50 | 8.33 |
| | 11) | | |
| 5. | Sodium Alginate (Keltone HVCR) | 80 | 13.33 |
| 6. | Calcium carbonate (Heavy) | 56 | 9.33 |
| 7. | Magnesium stearate | 5 | 0.83 |
| 8. | Talc | 9 | 1.5 |
| 9. | Purified water | q.s | q.s |

Procedure: Same as mentioned in Example—2.

FIG. 1 shows the comparative dissolution profiles of the said invented tablet formulation and the reference formulation (Toprol XL Tablets).

### Example—4

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 29.23 |
| 2. | Lactose monohydrate | 60 | 9.23 |
| 3. | Methocel K15 M CR | 150 | 23.1 |
| 4. | Hydroxypropyl cellulose (HPC-M) | 100 | 15.38 |
| 5. | Sodium Alginate (Keltone HVCR) | 80 | 12.31 |
| 6. | Calcium carbonate (Heavy) | 56 | 8.62 |
| 7. | Magnesium stearate | 5 | 0.77 |
| 8. | Talc | 9 | 1.38 |
| 9. | Purified water | q.s | q.s |

Procedure: Same as mentioned in Example—2.

FIG. 1 shows the comparative dissolution profiles of the said invented tablet formulation and the reference formulation (Toprol XL Tablets).

### Example—5

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 28.15 |
| 2. | Lactose monohydrate | 60 | 8.89 |
| 3. | Methocel K15 M CR | 175 | 25.93 |
| 4. | Hydroxypropyl cellulose (HPC-L) | 100 | 14.81 |
| 5. | Sodium Alginate (Keltone HVCR) | 80 | 11.85 |
| 6. | Calcium carbonate (Heavy) | 56 | 8.3 |
| 7. | Magnesium stearate | 5 | 0.74 |
| 8. | Talc | 9 | 1.33 |
| 9. | Purified water | q.s | q.s |

Procedure: Same as mentioned in Example—2.

FIG. 1 shows the comparative dissolution profiles of the said invented tablet formulation and the reference formulation (Toprol XL Tablets).

### Example—6

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 27.14 |
| 2. | Lactose monohydrate | 60 | 8.57 |
| 3. | Methocel K15 M CR | 175 | 25 |
| 4. | Hydroxypropyl cellulose (HPC-L) | 125 | 17.86 |
| 5. | Sodium Alginate (Keltone HVCR) | 80 | 11.43 |
| 6. | Calcium carbonate (Heavy) | 56 | 8 |
| 7. | Magnesium stearate | 5 | 0.71 |
| 8. | Talc | 9 | 1.29 |
| 9. | Purified water | q.s | q.s |

Procedure: Same as mentioned in Example—2.

FIG. 1 shows the comparative dissolution profiles of the said invented tablet formulation and the reference formulation (Toprol XL Tablets).

### Example—7

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 27.14 |
| 2. | Lactose monohydrate | 60 | 8.57 |
| 3. | Methocel K15 M CR | 150 | 21.43 |
| 4. | Hydroxypropyl cellulose (HPC-L) | 150 | 21.43 |
| 5. | Sodium Alginate (Keltone HVCR) | 80 | 11.43 |
| 6. | Calcium carbonate (Heavy) | 56 | 8 |
| 7. | Magnesium stearate | 5 | 0.71 |
| 8. | Talc | 9 | 1.29 |
| 9. | Purified water | q.s | q.s |

Procedure: Same as mentioned in Example—2.

FIG. 1 shows the comparative dissolution profiles of the said invented tablet formulation and the reference formulation (Toprol XL Tablets).

### Example—8

Sustained release tablets were prepared using the following materials in the stated quantities:

| | | Quantity | Quantity |
|---|---|---|---|
| Sr No. | Ingredients | (mg/tablet) | (% w/w) |
| 1. | Metoprolol Succinate | 190 | 27.14 |
| 2. | Lactose monohydrate | 60 | 8.57 |
| 3. | Methocel K15 M CR | 150 | 21.43 |
| 4. | Hydroxypropyl cellulose (HPC-M) | 82 | 11.71 |
| 5. | Sodium Alginate (Keltone HVCR) | 120 | 17.14 |
| 6. | Calcium carbonate (Heavy) | 84 | 12 |
| 7. | Magnesium stearate | 5 | 0.71 |
| 8. | Talc | 9 | 1.29 |
| 9. | Purified water | q.s | q.s |

Procedure: Same as mentioned in Example—2.

FIG. 1 shows the comparative dissolution profiles of the said invented tablet formulation and the reference formulation (Toprol XL Tablets).

We claim:

1. An oral pharmaceutical composition comprising:
a therapeutically effective amount of metoprolol succinate or a pharmaceutically acceptable derivative thereof and a hydrophilic polymer matrix comprised of gelling agents which include at least one hydrophilic polymer with one or more gum or gum derivative and pharmaceutically acceptable excipients therefore wherein said composition provides a sustained or modified release of metoprolol succinate or a pharmaceutically acceptable derivative thereof.

2. The pharmaceutical composition of claim 1, wherein the said composition comprises metoprolol succinate or a pharmaceutically acceptable derivative thereof and a hydrophilic polymer matrix comprised of gelling agents comprising at least one hydrophilic polymer with one or more gum or gum derivatives mixed and compressed together.

3. The pharmaceutical composition of claim 2, wherein said hydrophilic polymer matrix comprises gelling agents comprising at least one hydrophilic polymer in combination with one or more gum or gum derivative.

4. The pharmaceutical composition of claim 3, wherein said hydrophilic polymer is selected from a group comprising cellulose polymers, cellulose alkyl hydroxylates, cellulose alkyl carboxylates and alkali metal salts of cellulose alkyl carboxylates, vinyl copolymers, polyethylene oxide and derivatives thereof, propylene glycol esters, carbomers and derivatives thereof, and polyvinyl pyrrolidones and derivatives thereof.

5. The pharmaceutical composition of claim 4 wherein the cellulose alkyl hydroxylate is selected from the group consisting of hydroxypropylmethyl cellulose, hydroxypropyl cellulose, hydroxymethyl cellulose, and hydroxyethyl cellulose.

6. The pharmaceutical composition of claim 4 wherein the cellulose alkyl carboxylate is selected from the group consisting of carboxymethyl cellulose, carboxyethyl cellulose and alkali metal salts thereof.

7. The pharmaceutical composition of claim 3, wherein said gum and gum derivative is a vegetable gum selected from the group consisting of alginates, xanthan gum, gum karaya, pectin, agar, tragacanth, acacia, carrageenan, tragacanth, chitosan, agar, alginic acid, other polysaccharide gums and mixtures thereof.

8. The pharmaceutical composition of claim 1, wherein said pharmaceutically acceptable diluents are selected from the group comprising lactose, mannitol, sucrose, dextrose, microcrystalline cellulose, powdered cellulose, starches, sorbitol, dibasic calcium phosphate, calcium carbonate and mixtures thereof.

9. The pharmaceutical composition of claim 1, wherein the composition is an oral solid dosage form.

10. The pharmaceutical composition of claim 9, wherein the composition is a capsule, tablet, granules, pills, granules in capsule, micro-tablets in capsules or combinations thereof.

11. The pharmaceutical composition of claim 1, wherein the composition is in tablet dosage form.

12. The pharmaceutical composition of claim 11, wherein the tablet comprises sustained released metoprolol succinate or a pharmaceutically acceptable derivative thereof with a hydrophilic polymer matrix mixed and compressed together and may optionally be coated with suitable coating materials.

13. The pharmaceutical composition of claim 1, wherein the composition is manufactured by a process comprising the steps of:
(i) mixing and blending metoprolol succinate or a pharmaceutically acceptable derivative thereof with a hydrophilic polymer matrix
(ii) granulating the product of step (i) using suitable granulation technique followed by
(iii) compressing the product of step (ii) to form tablets comprising metoprolol succinate.

14. The pharmaceutical composition of claim 13, wherein the tablet is manufactured by using wet granulation technique, drying the resultant product and compressing the dried product to form the tablets.

15. A process for manufacture of sustained release composition of metoprolol succinate or a pharmaceutically acceptable derivative thereof, the process comprising the steps of:
(a) mixing metoprolol succinate or a pharmaceutically acceptable derivative thereof with at least one hydrophilic polymer matrix
(b) granulating the product of step (a) using suitable granulation technique followed by
(c) compressing the product of step b to form tablets comprising metoprolol succinate or the pharmaceutically acceptable derivative thereof.

16. A pharmaceutical composition in solid dosage form prepared by the process of claim 15, wherein the process comprises blending metoprolol succinate or a pharmaceutically acceptable derivative thereof with gelling agents comprising hydrophilic polymer, a gum or gum derivatives and pharmaceutical acceptable diluents; granulating the same using aqueous solvents wherein the mixture may optionally include one or more binders; followed by drying the resultant product; optionally, lubricating the same using suitable lubricants and antiadherants and compressing the lubricated granules to form tablets containing metoprolol succinate or the pharmaceutically acceptable derivative thereof.

17. The pharmaceutical composition of claim 16, wherein the hydrophilic polymer is hydroxyl propyl cellulose and/or hydroxypropyl methyl cellulose.

18. The pharmaceutical composition of claim 16, wherein the gum or gum derivative is sodium alginate.

19. The pharmaceutical composition of claim 16, wherein the diluents are microcrystalline cellulose, lactose, calcium carbonate or mixtures thereof.

20. The pharmaceutical composition of claim 16, wherein the diluent is selected from the group consisting of cellulose derivatives, povidone, nonaqueous or hydroalcoholic solvents and mixtures thereof.

21. The pharmaceutical composition of claim 16 wherein the lubricants and antiadherants are magnesium stearate, talc or mixtures thereof.

22. The process of claim 15, wherein the tablet comprises metoprolol succinate or a pharmaceutically acceptable derivative thereof and a hydrophilic polymer matrix comprising gelling agents comprising at least one hydrophilic polymer in combination with gum or gum derivatives.

23. The process of claim 15, wherein the hydrophilic polymer matrix comprises gelling agents comprising at least one hydrophilic polymer with a gum or gum derivatives.

24. The process of claim 15, wherein the hydrophilic polymer is selected from a group comprising cellulose polymers selected from the group consisting of cellulose ethers, cellulose alkyl hydroxylates, and cellulose alkyl carboxylates;, vinyl copolymers, polyethylene oxide and derivatives thereof, propylene glycol esters, carbomers and derivatives thereof, polyvinyl pyrrolidones and derivatives thereof.

25. The process of claim 24, wherein the cellulose alkyl hydroxylate is selected from the group consisting of hydroxypropyl methyl cellulose, hydroxypropyl cellulose, hydroxymethyl cellulose, hydroxy ethyl cellulose and mixtures thereof.

26. The process of claim 24, wherein the cellulose alkyl carboxylate is selected from the group consisting of carboxymethyl cellulose, carboxyethyl cellulose alkali metal salts thereof and mixtures thereof.

27. The process of claim 15, wherein the said gum and gum derivative is a vegetable gum.

28. The process of claim 27, wherein the vegetable gum is selected from the group consisting of alginates, xanthan gum, gum karaya, pectin, agar, tragacanth, acacia, carrageenan, tragacanth, chitosan, agar, alginic acid, other polysaccharide gums and mixtures thereof.

29. The process of claim 15, wherein the resulting composition is an oral solid dosage form.

30. The process of claim 29, wherein the oral solid dosage form is selected from the group consisting of a capsule, tablet, granules, pills, granules-in-capsule, micro-tablets-in-capsules and combinations thereof.

31. The process of claim 29, wherein the resulting composition is a tablet.

32. The process of claim 31, wherein the tablet is optionally coated.

33. The process of claim 31, wherein the tablet is manufactured by using a suitable granulation technique.

34. The process of claim 33, wherein the tablet is manufactured by using wet granulation technique.

35. A method of treating hypertension and related cardiac disorders in a subject in need of said treatment, which method comprises administering to the subject a pharmaceutical product or formulation according to claim 1.
